(19) United States
(12) Patent Application Publication
(10) Pub. No.: US 2023/0233222 A1
(43) Pub. Date: Jul. 27, 2023

(54) ANISOTROPIC SURGICAL INSTRUMENTS AND METHODS FOR PRODUCING SAME

(71) Applicant: DePuy Synthes Products, Inc., Raynham, MA (US)

(72) Inventor: Peyton L. Hopson, Jacksonville, FL (US)

(21) Appl. No.: 18/160,348

(22) Filed: Jan. 27, 2023

Related U.S. Application Data
(60) Provisional application No. 63/303,540, filed on Jan. 27, 2022.

(51) Int. Cl. A61B 17/29

(52) U.S. Cl. CPC A61B 17/29; A61B 2017/2903; A61B 2017/2901; A61B 2017/2926

(57) ABSTRACT

An anisotropic surgical instrument is provided having at least one reinforced region that includes at least one reinforcement fiber oriented along a direction that corresponds to a direction of the force applied to the surgical instrument during use. The instrument can further include a weakened region that defines a hinge about which the instrument can iterate between a contracted configuration and an expanded configuration. Methods of fabricating such instruments are also described.

### CROSS-REFERENCE TO RELATED APPLICATIONS

This claims priority to U.S. Patent Application Ser. No. 63/303,540 filed Jan. 27, 2022, the disclosure of which is hereby incorporated by reference as if set forth in its entirety herein.

### BACKGROUND

Surgical instruments are used in a large number of surgical applications, including but not limited to implant insertion, implant fixation, fracture reduction, vertebral distractions, bone repositioning, bone drills, and the like. Such instruments are conventionally often made from a biocompatible metal such as titanium or alloys thereof, or stainless steel. Some surgical instruments can be polymeric. Irrespective of the material, conventional surgical instruments are manufactured as isotropic structures that are designed to absorb forces, or loads, equally in all directions during use. As a result, conventional surgical instruments are sized to maintain their structural integrity in response to forces applied to the instrument in all directions. This results in geometric design limitations, and often results in large instrument sizes in order to accommodate the force requirements. However, the increased sizes are commonly associated with greater invasiveness of the soft tissue and other drawbacks.

What is therefore needed is an improved surgical instrument that suitably absorbs forces applied to the instrument during use.

### SUMMARY

In accordance with one example, an anisotropic surgical instrument can include a first reinforced region including a first plurality of reinforcement strands that are embedded in a solidified polymer, and a weakened region that includes the solidified polymer and one of i) none of the first plurality of reinforcement strands embedded in the solidified polymer; and ii) a density of the first plurality of strands less than that of the first reinforced region.

### BRIEF DESCRIPTION OF THE DRAWINGS

FIG. 1 is a perspective view of an anisotropic surgical reamer constructed in accordance with another aspect of the present invention;

FIG. 2 is a side elevation view of an anisotropic bone screw driving instrument in one example;

FIG. 3A is a perspective view of an anisotropic surgical retractor constructed in accordance with one aspect of the present invention shown in an initial position;

FIG. 3B is a perspective view of the surgical retractor of FIG. 3A, shown in a retracted position;

FIG. 4 is a perspective view of an anisotropic surgical impactor in accordance with another aspect of the present invention;

FIG. 5A is a schematic view of an anisotropic surgical instrument body having a plurality of reinforced regions constructed in accordance with one example, shown in an expanded configuration;

FIG. 5B is a schematic view of the anisotropic surgical instrument body of FIG. 5A, shown in a contracted configuration;

FIG. 5C is a schematic sectional view of an instrument assembly including the anisotropic surgical instrument body of FIG. 5A and an outer sleeve inserted into a patient;

FIG. 6A is a schematic view of an anisotropic surgical instrument body having a plurality of reinforced regions constructed in accordance with another example, shown in an expanded configuration;

FIG. 6B is a schematic view of the anisotropic surgical instrument body of FIG. 6A, shown in a contracted configuration;

FIG. 7 is a perspective view of an anisotropic anatomical implant configured as an anisotropic bone plate fixed to an underlying bone in another example;

FIG. 8A is a perspective view of an anisotropic anatomical implant configured as an anisotropic knee prosthesis in another example; \

FIG. 8B is an exploded perspective view of the anisotropic anatomical implant of FIG. 8A; and

FIG. 9 is a perspective view showing a method of constructing an anisotropic surgical instrument.

### DETAILED DESCRIPTION

Throughout the description below, reference is made to singular elements using terms such as “a,” “an,” or “the.” However, it is recognized that singular elements can also apply to plural elements or at least one of the elements. Thus, description below of a singular element applies with equal force and effect to a plurality of elements, including at least one of the elements. Conversely, reference to a plurality of elements can also apply with equal force and effect to a single one of the elements or at least one of the elements. Further, description of at least one element can apply with equal force and effect to a singular element or a plurality of the elements.

In accordance with certain aspects of the present invention, anisotropic surgical implements and methods for fabricating and using same are described. The anisotropic surgical implements can include anisotropic surgical instruments and anisotropic anatomical implants. The anisotropic surgical implements can include various bodies made from a solidified material, such as a polymer. The bodies can include one or more reinforced regions having reinforcement strands that can be oriented in respective directions that accommodate the anticipated load direction and load magnitude during use at the reinforced regions.

In this regard, the anisotropic surgical instruments can be configured to apply a force, or load, to an anatomical structure or implant during use, and thus are configured to receive a corresponding counterforce. The reinforcement strands can be oriented substantially along a direction of the counterforce so as to strengthen the anisotropic surgical instruments during use. The anisotropic surgical instruments can further include one or more weakened regions adjacent one or more of the reinforced regions. The weakened regions can define hinges when it is desired to move the instrument between contracted and expanded configurations. In some examples, the instruments are configured such that forces designed to be applied to the instruments during use do not cause the instruments to move from the expanded configuration to the contracted configuration. Various instruments will now be described as being anisotropic in accordance with certain examples. However, it should be appreciated that any suitable surgical instrument that is configured to apply a force to an anatomical structure or anatomical implant, and thus is configured to receive a corresponding counterforce, can be constructed as an anisotropic surgical instrument as described herein, and all such instruments are contemplated herein.

The anisotropic anatomical implants can be configured to receive a force, or load, along a direction during use, and the reinforcement strands can be oriented substantially along the direction of the load so as to strengthen the anisotropic anatomical implants during use. Further, when the implant define a bone fixation implant such as a bone plate, the direction of the load can be substantially aligned with the loading on the bone during normal anatomical function. When the implant defines one or more articulating components of an anatomical joint implant, the reinforcement strands can be oriented along a direction that is in-plane with the direction of articulation of the implant. Further, the implant can define one or more weakened regions and one or more reinforced regions that have a greater density of reinforcement strands than the weakened region. The one or more reinforced regions can be disposed at regions of bone having high bone density, and the one or more weakened regions can be disposed at regions of bone having lower bone density that is less than the high bone density. The weakened regions can include reinforcement strands of less density than the strands of the reinforced regions, or can be devoid entirely of reinforcement strands. Furthermore, the reinforcement strands can cause the resulting implant to match the modulus and strength of the bone. It should be appreciated that any suitable anatomical implant that is configured receive a load can be constructed as an anisotropic anatomical implant as described herein, and all such implants are contemplated herein.

Unless otherwise indicated, the term “substantially” as used herein can refer to manufacturing tolerances. In one example, the term “substantially” as used with reference to a size, shape, direction, or other parameter can equal the stated size, shape, direction, or other parameter as well as +/−20% of the size, shape, direction, or other parameter can equal the stated size, shape, direction, or other parameter, including +/−18% of the size, shape, direction, or other parameter can equal the stated size, shape, direction, or other parameter, including +/−16% of the size, shape, direction, or other parameter can equal the stated size, shape, direction, or other parameter, including +/−14% of the size, shape, direction, or other parameter can equal the stated size, shape, direction, or other parameter, including +/−12% of the size, shape, direction, or other parameter can equal the stated size, shape, direction, or other parameter, including +/−10% of the size, shape, direction, or other parameter can equal the stated size, shape, direction, or other parameter, including +/−8% of the size, shape, direction, or other parameter can equal the stated size, shape, direction, or other parameter, including +/−6% of the size, shape, direction, or other parameter can equal the stated size, shape, direction, or other parameter, including +/−4% of the size, shape, direction, or other parameter can equal the stated size, shape, direction, or other parameter, including +/−2% of the size, shape, direction, or other parameter can equal the stated size, shape, direction, or other parameter, including +/−1% of the size, shape, direction, or other parameter can equal the stated size, shape, direction, or other parameter.

Referring now to FIG. 1 an anisotropic surgical instrument 20 in one example can be configured as a reamer 22. The anisotropic surgical instrument 20 includes an instrument body 48 that can include a shaft 24 of the reamer 22. The shaft 24 is elongate along a central axis 26. The shaft 24 has a proximal end 28 and a distal end 30 that is opposite the proximal end 28 in a distal direction. The distal direction can be oriented along the central axis 26. The shaft 24 can be substantially cylindrical or alternatively shaped as desired. The instrument body 48 of the anisotropic surgical instrument 20 can further include a head 32 of the reamer 22 that extends in the distal direction from the distal end 30 of the shaft 24. The head 32 can be attached to the shaft 24 or can be monolithic with the shaft 24 as desired.

The head 32 can define a torsional driving portion of the reamer 22. In one example, the head 32 can be configured as a reamer head 34. The reamer head 34 can include a plurality of radially extending blades 36 that each define an angled cut portion 40. The reamer head 34 can be configured to rotate about the central axis 26 at a sufficient speed that causes the blades 36, and in particular the angled cut portions 40, to mill or otherwise ream out a target structure. The target structure can be configured as an anatomical structure, such as one or more bones, or an anatomical implant.

In one example, the shaft 24 can be rotatable about the central axis 26, which thus causes the reamer head 32 to rotate about the central axis 26. For instance, the shaft 24 includes a proximal coupling 42 at the proximal end 28 that is configured to be rotatably coupled to a drive. Thus, the proximal coupling 42 can define a handle portion of the shaft 24. During use, the drive can impart a torsional force to the shaft 24 about the central axis 26 that causes the shaft 24, and thus the reamer head 34 to rotate about the central axis 26 at a speed sufficient for the reamer head 34 to mill or otherwise ream out the target structure. For instance, the proximal coupling 42 can be received by the drive so as to rotatably couple the shaft 24 to the drive. As shown, the coupling 42 can define a recess or groove 44 that is configured to be engaged by a projection of the drive or extension to thereby secure the reamer 18 to the drive. The drive may be a manually operated drive or a drive powered by a battery or alternative power source.

The reamer 22 can further define a bore 46 that extends through the shaft 24 and the reamer head 34 along the central axis 26. The bore 46 is configured to receive a temporary fixation element, such as a Kirschner wire, or K-wire, that extends from the target structure that is to be reamed. Therefore, the bore 46 may have a diameter that is substantially equal to the diameter of the temporary fixation element.

It should be appreciated that during use, forces can be applied to the reamer 22. For instance, the proximal end 28 can receive first torsional forces from the drive. The first torsional forces can be oriented about the central axis 26 in a first helical direction that include a rotational component substantially about the central axis 26 and an axial component substantially along the central axis 26. Forces can also be applied from the reamer head 34 to the target structure in the first helical direction. The target structure can thus apply a helical counterforce to the reamer 22 that defines a second helical force oriented about the central axis in a second helical direction that is opposite the first helical direction. A majority of the second helical force can be disposed at the reamer head 34 and the distal end 30. The shaft 24 can further receive axial forces along a longitudinal direction that defines the central axis 26 as the reamer 22 is urged axially against the target structure.

Thus, it should be appreciated that when the anisotropic surgical instrument 20 is configured as the reamer 22, the instrument body 48 of the anisotropic surgical instrument 20 receives the first and second helical forces. The instrument body 48 can further receive the axial forces. The instrument body 48 can be defined by either or both of the shaft 24 and the reamer head 34 as described above. As will be appreciated from the description below, the instrument body 48 of the surgical instrument can define reinforced regions that are configured to absorb the forces during use.

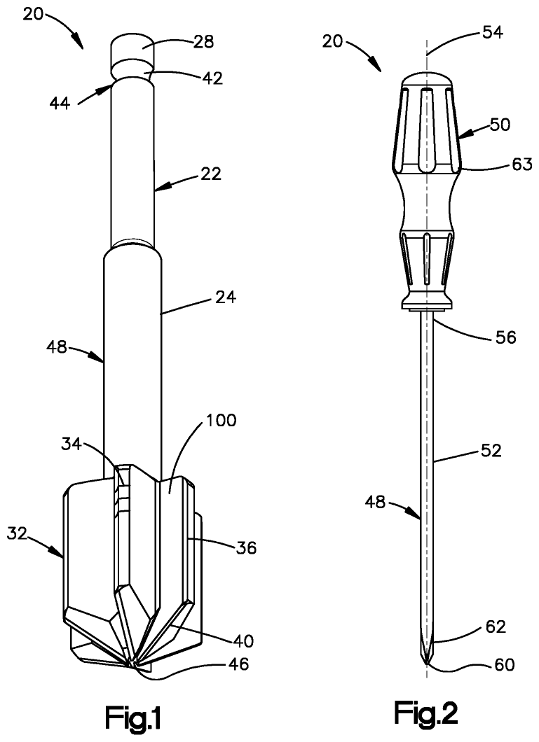

As will now be described, the anisotropic surgical instrument 20 can define other instrument configurations that generate forces on the instrument body 48 during use. For instance, as shown in FIG. 2, the anisotropic surgical instrument 20 can be configured as a bone screwdriver 50. The instrument body 48 of the surgical instrument 20 can include a shaft 52 of the screwdriver 50 that is elongate along a central axis 54. The shaft 52 has a proximal end 56 and a distal end 58 that is opposite the proximal end 56 in a distal direction. The distal direction can be oriented along the central axis 54. The shaft 52 can be substantially cylindrical or alternatively shaped as desired. The instrument body 48 of the surgical instrument 20 can include a head 60 of the screwdriver 50 that extends in the distal direction from the distal end 58 of the shaft 52. The head 60 can be attached to the shaft 52 or can be monolithic with the shaft 52 as desired.

The head 60 can define a torsional driving portion of the screwdriver 50. In one example, the head 60 can be configured as a screw driving head 62. The screw driving head 62 can be configured as a flat head, hex head, a cruciform, or a socket as desired that is configured to rotatably couple to a bone screw. The screwdriver 50 can include a handle 63 at the proximal end 56 that receives a manual torsional force that urges the shaft 52 and screw driving head 62 to rotate about the central axis 54. Alternatively, the proximal end 56 can be coupled to a driver that can provides a torsional force urge the shaft 52 and screw driving head 52 to rotate about the central axis 54. Rotation of the screw driving head 62 about the central axis 54 in a first direction causes the screw driving head 62 to rotate the bone screw in a direction that drives the bone screw into a target anatomical structure, such as one or more bones. Rotation of the screw driving head 62 about the central axis 54 in a second direction opposite the first direction causes the screw driving head 62 to rotate the bone screw in a direction that removes the bone screw from the target anatomical structure.

It should be appreciated that during use, forces can be applied to the screwdriver 50. For instance, the proximal end 56 and/or the handle 63 can receive first torsional forces that can be manual or received from a drive. The first torsional forces can be oriented about the central axis 54 in a first helical direction. Forces can also be applied from the screw driving head 62 to the target structure in the first helical direction. The target structure can thus apply a helical counterforce to the screw driving head 62 that defines a second helical force oriented about the central axis in a second helical direction that is opposite the first helical direction. A majority of the second helical force can be disposed at the screw driving head 62 and the distal end 58. The shaft 52 can further receive axial forces along a longitudinal direction that defines the central axis as the screwdriver 50 is urged axially against the bone screw.

Thus, it should be appreciated that when the anisotropic surgical instrument 20 is configured as the screwdriver 50, the instrument body 48 of the anisotropic surgical instrument 20 receives the first and second helical forces. The instrument body 48 can further receive the axial forces. The instrument body 48 can be defined by one or more up to all of the shaft 52, the handle 63, and the screwdriving head 62 as described above. As will be appreciated from the description below, the instrument body 48 of the surgical instrument can define reinforced regions that are configured to absorb the forces during use.

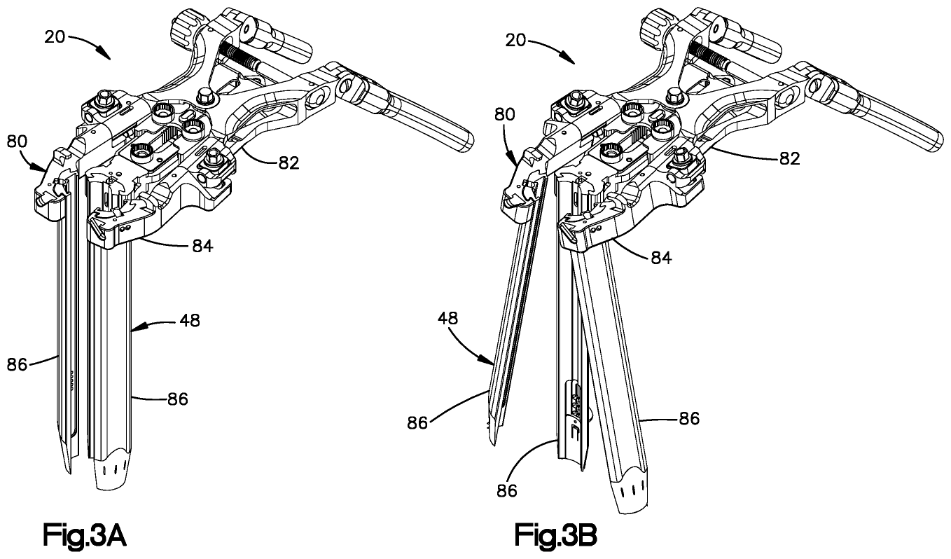

Referring now to FIG. 3A-3B, the anisotropic surgical instrument 20 can be configured as a surgical access retractor 80 that is configured to dilate soft tissue to create an access path to a target surgical site. The retractor 80 can generally include a handle assembly 82 and a support assembly 84 that supports the handle assembly 82. The retractor 80 can further include one or more retractor arms 86 that are coupled to the support assembly 84 and extend generally distally from the support assembly 84. The retractor 80 can include any number of retractor arms 86, such as two, three, or more as desired. One or more up to all of the retractor arms 86 can be translatable and/or pivot with respect to the other retractor arms 86 so as to move away from the other retractor arms 86 from a first or contracted configuration shown in FIG. 3A to an expanded configuration shown in FIG. 3B, thereby dilating the surrounding soft tissue. It should thus be appreciated that the soft tissue can apply a torsional force to the retractor arms 86 which can be communicated to the support assembly 84. The instrument body 48 of the anisotropic surgical instrument 20 can thus be defined by either or both of the retractor arms 86 and the support assembly 84.

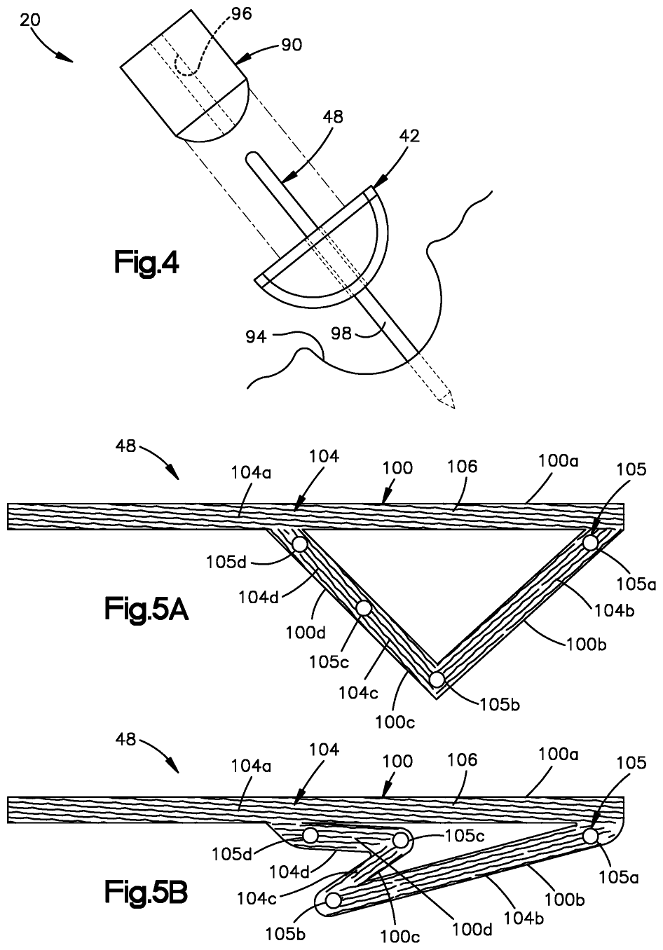

Referring now to FIG. 4, the anisotropic surgical instrument 20 can be configured as a surgical impaction instrument 90 that is configured to deliver impaction forces so as to drive an implant 92 into an anatomical site 94. The anatomical site 94 can be a bone 96, or can alternatively be an intervertebral disc space, a bone, soft tissue, or the like. The impaction instrument 90 can be sized and shaped to correspond to a geometry of the implant 92. The impaction instrument 90 can include a passageway 96 extends therethrough along a longitudinal direction. The passageway 96 can be sized to slidably receive a guide member 98 that can be temporarily implanted into the anatomical site 94. The guide member 98 can be configured as a Kirschner wire, or K-wire. When the impaction instrument 90 receives the guide member 98 in the passageway 96, the impaction instrument 90 is movable along the guide member 98 in a distal direction to the implant 92 so as to drive the implant 92 into the anatomical site 94. The distal direction can be oriented along the longitudinal direction. In some examples, the impaction instrument 90 can be driven with a hammer or other suitable drive instrument against the implant 92. The impaction instrument 90 can impact the implant 92 as many times as desired until the implant 92 is seated in the anatomical site 94.

It should be appreciated that the impaction instrument 90 receives forces along the longitudinal direction during use. In particular, the drive instrument applies an impaction force in the distal direction to a proximal end of the impaction instrument 90. The implant 92 applies a counterforce to a distal end of the impaction instrument 90 that is opposite the proximal end in the distal direction. The impaction force from the drive instrument can be oriented in the distal direction. The counterforce from the implant 92 can be oriented in a proximal direction that is opposite the distal direction. The proximal and distal directions can be oriented along the longitudinal direction. Accordingly, the impaction force and the counterforce can be substantially straight and linear forces.

Accordingly, when the anisotropic surgical instrument 20 is configured as the impaction instrument 90, the instrument body 48 of the anisotropic surgical instrument 20 receives the impaction force and the counterforce. The instrument body 48 can be defined by the impaction instrument 90 as described above. As will be appreciated from the description below, the instrument body 48 of the surgical instrument can define reinforced regions that are configured to absorb the forces during use.

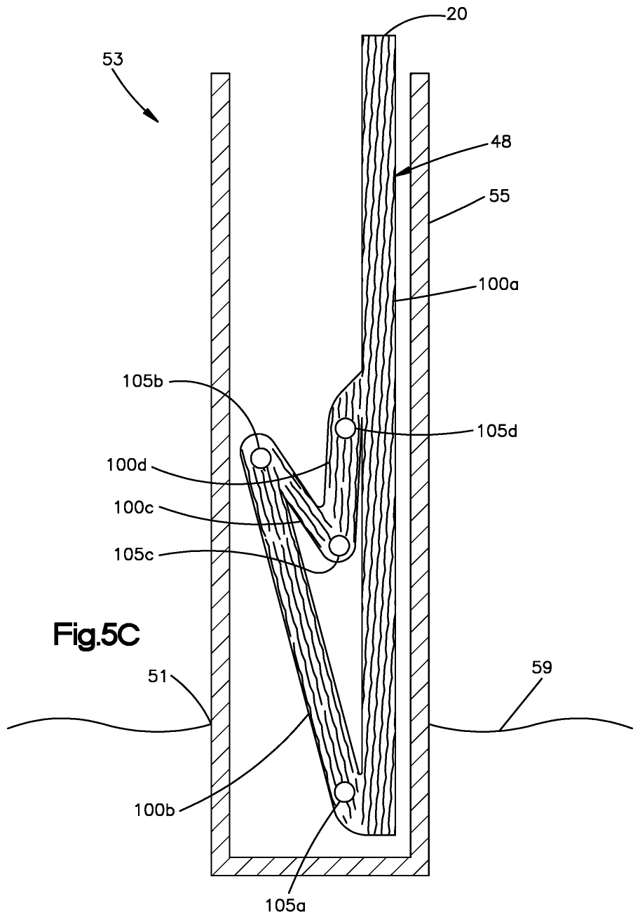

Referring now to FIGS. 5A-5B, the instrument body 48 of the anisotropic surgical instrument 20 is schematically shown in accordance with one example. The anisotropic surgical instrument 20 can be configured as described above or can define any suitable alternative instrument, and the instrument body 48 can be configured to absorb forces that are applied to the instrument body 48 during use. In particular, the instrument body 48 can include at least one anisotropic reinforced region 100 that is configured to absorb the forces applied to the instrument body 48 during use. Each reinforced region 100 can include a resin 102 (see FIG. 9) that has been solidified so as to define a polymer 106, and at least one reinforcement strand 104 such as a plurality of reinforcement strands 104 that are disposed in the polymer 106. For instance, the reinforcement strands 104 can be embedded in the polymer 106. The reinforcement strands 104 can define fibers and can thus be referred to as fibrous reinforcement strands. The reinforcement strands 104 can be carbon strands, glass strands, or strands of any suitable alternative material as desired. Alternatively, the reinforcement strands 104 can be defined by glass beads.

The reinforcement strands 104 can be oriented substantially along a respective direction that is substantially oriented along a direction of a force that is applied to the instrument body 48 during use. In one example, the force can be a primary force, meaning no other force applied to the reinforced region has a greater magnitude than the primary force. In other examples, it may be desired to orient the reinforcement strands substantially along a direction that is oriented along a direction of a force applied to the reinforced region 100 having a magnitude less than that of the primary force. The instrument body 48 can further include one or more weakened regions 105 disposed between adjacent ones of the reinforced regions 100. The weakened region 105 can define a hinge that allows the anisotropic anatomical implant 20 to move between an expanded configuration shown in FIG. 5A and a contracted configuration shown in FIG. 5B. The instrument body 48 will now be described in more detail.

The instrument body 48 of the anisotropic surgical instrument 20 can include any number of reinforced regions 100 as desired, depending on how many different forces are going to be applied to the instrument body 48 during use, and how many sections of the instrument body 48 are going to experience such forces during use. In one example, the instrument body 48 includes a first reinforced region 100a having a first plurality of reinforcement strands 104a, a second reinforced region 100b having a second plurality of reinforcement strands 104b, a third reinforced region 100c having a third plurality of reinforcement strands 104c, and a fourth reinforced region 100d having a fourth plurality of reinforcement strands 104d. It should be appreciated that a given anisotropic surgical instrument 20, including the instrument body 48, can include greater or fewer reinforced regions as desired.

Each of the reinforced regions 100 can be spaced in their respective entireties from the others of the reinforced regions 100. Alternatively, one or more of the reinforced regions 100 can overlap with one or more others of the reinforced regions 100. In the illustrated configuration, the second reinforced region 100b is disposed adjacent the first reinforced region 100a. In the illustrated configuration, the third reinforced region 100c is disposed adjacent the second reinforced region 100b. In the illustrated configuration, the fourth reinforced region 100d is disposed adjacent the third reinforced region 100c. In the illustrated configuration, the fourth reinforced region 100d is further disposed adjacent the first reinforced region 100a. This configuration can change during use, and is illustrated to depict one embodiment of body that is reinforced to absorb forces of different directions, and is movable between the contracted and expanded configurations. While the instrument body 48 can include four reinforced regions as shown, it should be appreciated that the body can include any number of reinforced regions, such as at least one up to any number as desired depending on the forces to be applied to the instrument body 48 and absorbed by respective reinforced regions during use.

The instrument body 48 can be made from any suitable solidified polymer 106, and can define the plurality of reinforced regions 100. The first reinforced region 100a can include a first plurality of reinforcement strands 104a that are embedded in the solidified polymer 106. Similarly, the second reinforced region 100b can include a second plurality of reinforcement strands 104b that are embedded in the solidified polymer 106. The third reinforced region 100c can include a third plurality of reinforcement strands 104c that are embedded in the solidified polymer 106. The fourth reinforced region 100d can include a fourth plurality of reinforcement strands 104d that are embedded in the solidified polymer 106.

Each reinforced region 100 can be a directionally reinforced region whereby the reinforcement strands 104 are oriented substantially along a respective direction. In one example, the reinforcement strands 104 can be oriented substantially inline with respective forces that are applied to the reinforced region 100 during use of the surgical instrument 20. Thus, the reinforcement strands 104 of each reinforced region 100 can be substantially inline with each other. In one example, the reinforcement strands 104 can be oriented substantially straight and linear so as to absorb straight and linear forces during use. In this regard, it is recognized that each individual reinforcement strand 104 of each reinforced region 100 need not extend entirely straight and linearly. However, the aggregate of the reinforcement strands 104 of each reinforced region 100 on average extend substantially straight and linearly as would be understood by one having ordinary skill in the art. Further, the strands 104 of each reinforced region 100 can be oriented substantially parallel to each other.

It should therefore be appreciated that each of the reinforced regions 100 can include a respective plurality of reinforcement strands 104 that extend in a respective direction. In this regard, the reinforced regions 100 can be referred to as directionally reinforced regions 100 whose reinforcement strands 104 are oriented in a respective direction. For instance, the first reinforcement strands 104a can be oriented substantially along a first direction. The second reinforcement strands 104b can be oriented substantially along a second direction. The second direction can be substantially the same as the first direction. Alternatively, the second direction can be different than the first direction. The third reinforcement strands 104c can be oriented substantially along a third direction. The third direction can be substantially the same as either or both of the first and second directions. Alternatively or additionally, the third direction can be different than either or both of the first and second directions. The fourth reinforcement strands 104d can be oriented substantially along a fourth direction. The fourth direction can be substantially the same as one or more up to all of the first, second, and third directions. Alternatively or additionally, the fourth direction can be different than one or more up to all of the first, second, and third directions. Different directions can be angled with respect to each other by at least 20 degrees, such as at least 30 degrees, such as at least 45 degrees. In other examples, at least one of the first, second, third, and fourth directions can have a different directional characteristic from at least one other of the first, second, third, and fourth directions. For instance, one directional characteristic can be a substantially straight and linear direction. Another directional characteristic can be a substantially curved direction, such as a helical or circular orientation. Other directional characteristics can define any suitable geometry as desired so as to provide a directional reinforcement along the direction of the directional characteristic.

While each reinforced region 100 can include a plurality of reinforcement strands 104 that are oriented in the respective directions, it is appreciated that each reinforced region 100 can alternatively include a single reinforcement strand 104. The single reinforcement strand 104 can extend along a single path substantially in the respective direction. Alternatively, the single reinforcement strand 104 can be folded about itself as many times as desired so as to extend back and forth along the several paths in respective direction as desired. Thus, each reinforced region 100 can be said to include at least one reinforcement strand 104. The at least one reinforcement strand 104 is present in sufficient quantity to provide for the increased strength characteristics as described herein. In one example, the at least one reinforcement strand 104 can be present in each reinforced region 100 in a range from approximately 10% to approximately 80% by volume, such as from approximately 25% to approximately 70% by volume, including from approximately 35% to approximately 65% by volume.

It is recognized that while the reinforced regions 100 can include the reinforcement strands 104 that are oriented substantially in the direction of the load during use, the reinforced regions 100 can include additional auxiliary strands in addition to the reinforcement strands as desired. Alternatively, the reinforcement strands 104 can be the only strands at the reinforced region 100, such that the reinforced region is devoid of auxiliary strands. In examples whereby the reinforced region 100 includes auxiliary strands, the auxiliary strands can also be embedded in the solidified polymer 106, and can be oriented in any direction as desired. The auxiliary strands can be made of the same material or different material than the reinforcement strands 104. Further, the auxiliary strands can have any suitable shape as desired. It may be desired to include auxiliary strands, for instance, if it is anticipated that multiple forces in different directions can be applied to the reinforced region 100. A strand can be considered to be an auxiliary strand if it is angularly offset at least 20 degrees with respect to the reinforcement strands. In one example, less than half of all strands in each of the reinforced regions 100 are oriented along a direction that is at least 20 degrees angularly offset with respect to the respective direction of the reinforcement strands 104. For instance, in some examples, less than 25% of all strands in each of the reinforced regions 100 are oriented along a direction that is at least 20 degrees angularly offset with respect to the respective direction of the reinforcement strands 104.

With continuing reference to FIGS. 5A-5B, the instrument body 48 can include one or more weakened regions 105. The weakened regions 105 can extend between different reinforced regions 100. For instance, the weakened region 105 can extend from one of the reinforced regions 100 to another of the reinforced regions 100. Thus, a first weakened region 105a can extend from the first reinforced region 100a to the second reinforced region 100b. A second weakened region 105b can extend from the second reinforced region 100b to the third reinforced region 100c. A third weakened region 105c can extend from the third reinforced region 100c to the fourth reinforced region 100d. A fourth weakened region 105d can extend from the fourth reinforced region 100d to the first reinforced region 100a.

Each weakened region 105 can include the solidified polymer 106. In this regard, the solidified polymer 106 can define a monolithic unitary structure that extends along all reinforced regions 100 and all weakened regions 105. In one example, one or more up to all of the weakened regions 105 is devoid of the reinforcement strands 104 of the reinforced regions 100. For instance, the weakened regions 105 can be devoid of the reinforcement strands 104 of the reinforced regions 100 along an entirety of the cross-section of the instrument body 48 for a length of at least one-fourth of the length of an adjacent one of the reinforced regions 100 In one example, one or more up to all of the weakened regions 105 can also be devoid of auxiliary strands of the reinforced regions 100. For instance, the weakened regions 105 can be devoid of the auxiliary strands along an entirety of the cross-section of the instrument body 48 for a length of at least one-fourth of the length of an adjacent one of the reinforced regions 100. Alternatively, it is recognized that one or more up to all of the weakened regions 105 can include either or both of a number of reinforcement strands 104 and a number of auxiliary strands as desired. However, the density of the reinforcement strands 104 at the weakened regions can be less than that of the reinforced regions 100. For instance, the weakened regions 105 can have a density of reinforcement strands 104 that is less than that of an adjacent reinforced region 100 along an entirety of the cross-section of the instrument body 48 for a length of at least one-fourth of the length of the adjacent reinforced regions 100. In one example, the density of the reinforcement strands 104 at each weakened region 105 can be no more than 75%, including no more than 50%, including no more than 25%, of the density of the reinforcement strands 104 of each reinforced region 100 that is adjacent the weakened region 105.

The weakened regions 105 can be disposed at locations of the instrument body 48 that are unaffected by forces that are applied to the instrument body 48 during use. For instance, in one example, the weakened region either does not receive the forces during use, or receives less than 75% of the forces, such as less than 50% of the forces. Alternatively or additionally, the weakened regions 105 can be disposed at locations of the instrument body 48 that are designed to flex so as to define a hinge when it is desired to move the instrument body 48 from the contracted configuration to the expanded configuration, or from the expanded configuration to the contracted configuration. For instance, it may be desirable to move the instrument body 48 to the contracted configuration for storage, or for minimally invasive access to an anatomical structure. In particular, it should be appreciated that the footprint of the instrument body 48 can be reduced when the instrument body 48 is in the contracted configuration as compared to when the instrument body 48 is in the expanded configuration. Consequently, as illustrated at FIG. 5C, an incision 51 into the patent 59 can be reduced when the instrument body 48 is in the contracted configuration as compared to when the instrument body 48 is in the expanded configuration. An instrument assembly 53 can include a sleeve 55 that is configured to be inserted into the incision 51. The instrument body 48 can be driven through the sleeve 55 to the target structure in its contracted configuration, and subsequently expanded to the expanded configuration if desired. Expansion of the instrument body 48 can occur prior to or after removal of the sleeve 55.

The instrument body 48 can be expanded about one or more hinges to the expanded configuration so that the anisotropic anatomical implant 20 is configured for use. The reinforcement strands 104 can substantially maintain their orientation relative to the respective central axis of the corresponding reinforced region 100 as the instrument body 48 moves between the contracted configuration and the expanded configuration. The central axis can be defined by a centralized axis along which the corresponding reinforced region 100 is elongate. Further, the respective directions defined by the orientations of the reinforced regions 100 can angulate with respect to each other as the instrument body 48 moves between the expanded configuration and the contracted configuration. For instance, the first direction and the second direction can define a first angle when the instrument body 48 is in the expanded configuration, and can define a second angle that is different than the first angle when the instrument body 48 is in the contracted configuration.

With continuing reference to FIGS. 5A-5B, the instrument body 48 of the anisotropic surgical instrument can include any number of segments as desired. For instance, the instrument body 48 can include only one segment. In other examples, the instrument body 48 can include a plurality of segments. Adjacent ones of the segments can be separated by a respective hinge. The segments and the hinges can be monolithic and unitary with each other. In particular, the solidified polymer 106 of the weakened region 105 is monolithic and unitary with the solidified polymer 106 of the reinforced regions 100. The respective orientation of the reinforcement strands 104 can be in any suitable direction as desired. In one example, each of the reinforcement regions 100 can be defined by a respective segment of the instrument body 48 that is elongated along a respective central axis. The direction of the orientation of the reinforcement strands 104 can be parallel to the central axis in some examples. Alternatively, the direction of the orientation of the reinforcement strands 104 can be angularly offset with respect to the respective central axis. Alternatively, as will now be described, the direction of the orientation of the reinforcement strands 104 can be non-linear or non-straight as desired.

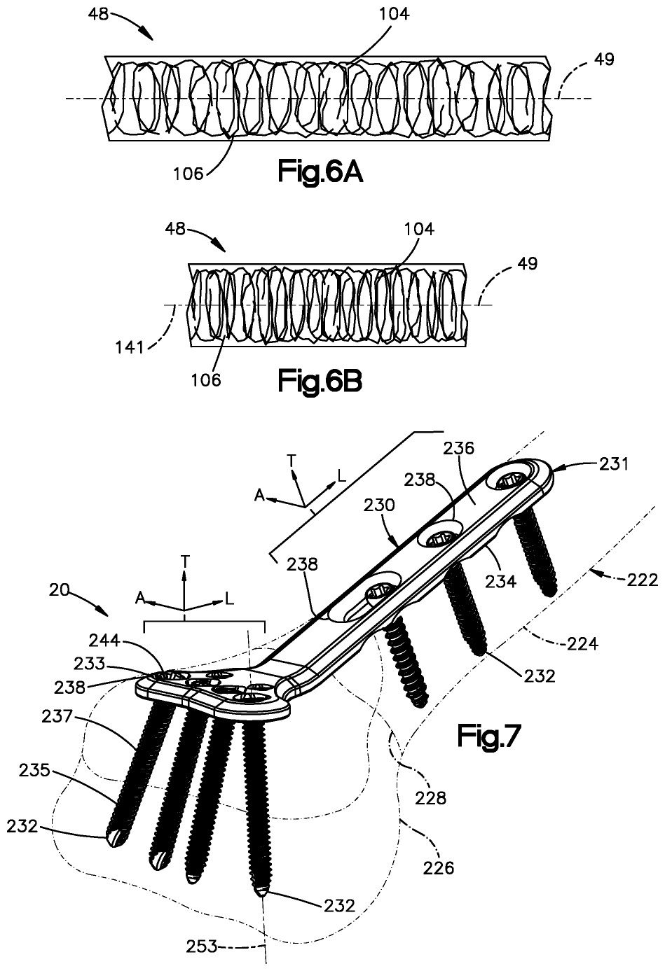

Referring now to FIGS. 6A-6B, the instrument body 48, or a section of the instrument body 48, can be elongate along a central axis 49. The orientation of the reinforcement strands 104 can be nonlinear. For instance, the reinforcement strands 104 can be curved, and oriented along a curved path. In one example, the reinforcement strands 104 can revolve about the central axis 49 so as to define successive revolutions about a respective axis of revolution 141. The axis of revolution 141 can be parallel with or defined by the central axis 49. Alternatively, the axis of revolution 141 can be angularly offset with respect to the central axis 49. The successive revolutions can be spaced from each other along the longitudinal direction that defines the central axis 49. The reinforcement strands 104 can extend substantially along a cylindrical path. The central axis 49 can define a center of the cylindrical path. Thus, the reinforcement strands 104 can be configured to absorb the torsional forces about the central axis 49 during use. In one example, the curved path of the reinforcement strands 104 can be a helical path. The reinforcement strands 104 can have substantially the same helical pitch or can have different helical pitches as desired. Alternatively, the reinforcement strands 104 can substantially define rings that extend about the central axis 49 along a plane that is perpendicular to the central axis 49.

When it is desired to move the instrument body 48 from the expanded configuration to the contracted configuration, a compressive force can be applied to the instrument body 48 along the central axis 49, which causes the longitudinal distance between the successive revolutions of the reinforcement strands 104 to decrease as illustrated in FIG. 6B. When it is desired to move the instrument body 48 from the contracted configuration to the expanded configuration, a tensile force can be applied to the instrument body 48 along the central axis 49, which causes the longitudinal distance between the successive revolutions of the reinforcement strands to increase as shown in FIG. 6A. Advantageously, the instrument body 48 can have a reduced length compared to conventional instruments that receive torsional forces, but can have sufficient torsional strength as provided by the reinforcement strands 104. It should be appreciated that the instrument body 48 can have high torsional strength and a flexural strength that is substantially lower than the torsional strength, such as no greater than 50% of the torsional strength.

As described above, the reinforced region 100 can be defined by a plurality of reinforcement strands 104, or can alternatively be defined by a single reinforcement strand 104. The single reinforcement strand 104 can define a single curved path along the central axis of revolution 141, or can be folded about itself and extend back and forth about the central axis of revolution 141 as desired. Thus, it can be said that the reinforced region 100 can be defined by at least one reinforcement strand 104 in the manner described above.

It should be recognized that any one or more up to all of the reinforced regions 100 can include strands 104 that are oriented along the curved path as desired. Further, any one or more up to all of the reinforced regions 100 of the instrument body 48 can include reinforcement strands 104 that extend substantially along the longitudinal direction as described above with respect to FIGS. 5A-5B in addition to reinforcement strands 104 that extend along the curved paths. In one example, the curved paths can surround the reinforcement strands 104 that substantially extend along the longitudinal direction.

Any one or more up to all of the reinforced regions 100 schematically illustrated in FIGS. 5A-6B can at least partially define the instrument body of any one of FIGS. 1-4 or any alternative instrument as desired. Thus, any one or more up to all of the reinforced regions 100 schematically illustrated in FIGS. 5A-6B can at least partially define the body of the reamer 22 of FIG. 1, the body of the bone screwdriver 50 of FIG. 2, and/or the surgical access retractor 80, or any other suitable surgical instrument as desired.

Referring now to FIG. 7, an anisotropic anatomical implant 220 can be configured to be implanted onto bone 222 so as to stabilize a first bone segment 224 with respect to a second bone segment 226 that is separated from the first bone segment 224 by a defect 228 such as a fracture. In one example, the first bone segment 224 can be defined by the diaphysis of the bone, while the second bone segment 226 can be defined by the metaphysis of the bone. It should be appreciated, however, that the first and second bone segments 224 and 226 can be defined by any region of the bone 222 as desired. Further, the bone 222 can be any bone in the human or animal anatomy suitable for bone plate fixation. Further still, while the bone 222 is illustrated having first and second bone segments 224 and 226, it is appreciated that the bone 222 can include any number of defects or bone fragments as desired that are configured for fixation using the implant 220. For instance, the diaphysis of the bone can include a plurality of bone fragments.

The implant 220 can be configured as a bone plate 230 that is configured to receive a plurality of bone anchors 232 so as to fix the bone plate 230 to the underlying bone 222, and in particular to each of the first and second bone segments 224 and 226. The bone anchors 232 include a head 233 and a shaft 235 that extends out with respect to the head 233 along a central anchor axis 253. The shaft 235 can extend directly from the head, or can extend from a neck that is disposed between the head 233 and the shaft 235. The shaft 235 can be threaded, such that the bone anchor 232 is configured as a bone screw 237 whose shaft 235 extends out relative to the head 233 along the central anchor axis 253, which can also be referred to as a central screw axis. The threaded shaft 235 can be configured to threadedly purchase in the underlying bone 222. For instance, one or more up to all of the bone screw 237 can be configured as a cortical screw whose threaded shaft 235 is designed and configured to threadedly mate to cortical bone. Alternatively or additionally, one or more of the bone screws 237 can be configured as a cancellous screw whose threaded shaft 235 is designed and configured to threadedly mate to cancellous bone. It is appreciated that cancellous bone screws have threads that have a greater pitch than threads of cortical bone screws. Further, the threads of cancellous bone screws typically extend out from the shaft of the bone screw a greater distance than the threads of cortical bone screws.

The implant 220 defines an implant body 260 that, in turn, defines an inner surface 234 configured to face the underlying bone 222, and an outer surface 236 that is opposite the inner surface 234 along a transverse direction T. The implant 220 further defines a plurality of fixation holes 238 that extend through the implant body 260 from the inner surface 234 to the outer surface 236. In particular, the implant body 260, and thus the bone plate 230, includes a plurality of internal surfaces 239 that extend from the outer surface 236 to the inner surface 234 and define a respective plurality of fixation holes 238 that extend from the outer surface 236 to the inner surface 234. The fixation holes 238 are sized to receive respective bone screws 237 that are configured to purchase with underlying bone.

The implant body 260, and thus the bone plate 230, can include a first plate portion 240 and a second plate portion 242. In one example, the first plate portion 240 can define a plate head portion that is configured to overlie the second bone segment 226, and the second plate portion 242 can be referred to as a plate shaft portion that is configured to overlie the first bone segment 224. Each of the first and second plate portions 240 and 242 can define a respective length that extends along a respective longitudinal direction L, a respective width that is less than the respective length and extends along a respective lateral direction A that is perpendicular to the respective longitudinal direction L, and a respective thickness that is less than both the respective length and the respective width and extends along the respective transverse direction T that is perpendicular to each of the respective longitudinal direction Land the respective lateral direction A.

As described above with respect to the instrument body 48, the implant body 260 can be configured to absorb forces that are applied to the implant 220 during use. In particular, the implant body 260 can include at least one anisotropic reinforced region 100 that is configured to absorb the forces applied to the implant body 260 during use. Each reinforced region 100 can include a resin 102 (see FIG. 9) that has been solidified so as to define a polymer 106, and at least one reinforcement strand 104 such as a plurality of reinforcement strands 104 that are disposed in the polymer 106. For instance, the reinforcement strands 104 can be embedded in the polymer 106. The reinforcement strands 104 of the implant body 260 are shown schematically in FIG. 7. The reinforcement strands 104 can define fibers and can thus be referred to as fibrous reinforcement strands. The reinforcement strands 104 can be carbon strands, glass strands, or strands of any suitable alternative material as desired. Alternatively, the reinforcement strands 104 can be defined by glass beads.

The reinforcement strands 104 can be oriented substantially along a respective direction that is substantially oriented along a direction of a load that is applied to the implant body 260 during use. The direction of the load can also be applied to the underlying bone during anatomical operation. For instance, the force can be a compressive force oriented along the longitudinal direction L. Therefore, the orientation of the reinforcement strands 104, can be along the longitudinal direction L, including at least one or both of the respective longitudinal direction L of the first plate portion 240 and the respective longitudinal direction L of the second plate portion 242. In one example, the force can be a primary force, meaning no other force applied to the reinforced region 100 has a greater magnitude than the primary force. In other examples, it may be desired to orient the reinforcement strands substantially along a direction that is oriented along a direction of a force applied to the reinforced region 100 having a magnitude less than that of the primary force.

The implant body 260 can further include at least one weakened region 105 that is disposed adjacent one of the reinforced regions 100. The weakened region 105 can be devoid of reinforcement strands 104 or can include a lower density of reinforcement strands 104 than the reinforced region 100. In one example, the density of the reinforcement strands 104 at the weakened region 105 can be no more than 75%, including no more than 50%, including no more than 25%, of the density of the reinforcement strands 104 of each reinforced region 100 that is adjacent the weakened region 105. The reinforced region 100 can be aligned with regions of the underlying bone that have high bone density, and thus high bone strength. The weakened region 104 can be aligned with a corresponding region of the underlying bone that has low bone density. The density of reinforcement strands 104 of the implant body 260 at each of the at least one reinforced region 100 and the weakened region 105 can be configured to match the modulus and strength of the bone. It should be appreciated that any suitable anatomical implant that is configured receive a load can be constructed as an anisotropic anatomical implant as described herein, and all such implants are contemplated herein.

The implant body 260 of the anisotropic anatomical implant 220 can include any number of reinforced regions 100 as desired, depending on how many different forces are going to be applied to the implant body 260 during use, and how many sections of the implant body 260 are going to experience such forces during use. In one example, the implant body 260 can include a first reinforced region 100a at one of the first and second plate portions 240 and 242, and a second reinforced region 100b at the other of the first and second plate portions 240 and 242. Each of the reinforced regions 100a and 100b can have respective pluralities of reinforcement strands 104 as described above. While the implant body 260 can include two reinforced regions as shown, it should be appreciated that the implant body 260 can include any number of reinforced regions, such as at least one up to any number as desired depending on the forces to be applied to the implant body 260 and absorbed by respective reinforced regions during use.

The implant body 260 can be made from any suitable solidified polymer 106, and can define the plurality of reinforced regions 100. The first reinforced region 100a can include a first plurality of reinforcement strands 104a that are embedded in the solidified polymer 106. Similarly, the second reinforced region 100b can include a second plurality of reinforcement strands 104b that are embedded in the solidified polymer 106.

Each reinforced region 100 can be a directionally reinforced region whereby the reinforcement strands 104 are oriented substantially along a respective direction. In one example, the reinforcement strands 104 can be oriented substantially inline with respective forces that are applied to the reinforced region 100 during use of the anatomical implant 220. Thus, the reinforcement strands 104 of each reinforced region 100 can be substantially inline with each other. In one example, the reinforcement strands 104 can be oriented substantially straight and linear so as to absorb straight and linear forces during use. In this regard, it is recognized that each individual reinforcement strand 104 of each reinforced region 100 need not extend entirely straight and linearly. However, the aggregate of the reinforcement strands 104 of each reinforced region 100 on average extend substantially straight and linearly as would be understood by one having ordinary skill in the art. Further, the strands 104 of each reinforced region 100 can be oriented substantially parallel to each other.

It should therefore be appreciated that each of the reinforced regions 100 can include a respective plurality of reinforcement strands 104 that extend in a respective direction. In this regard, the reinforced regions 100 can be referred to as directionally reinforced regions 100 whose reinforcement strands 104 are oriented in a respective direction. For instance, the first reinforcement strands 104a can be oriented substantially along a first direction. The second reinforcement strands 104b can be oriented substantially along a second direction. The second direction can be substantially the same as the first direction. Alternatively, the second direction can be different than the first direction. Different directions can be angled with respect to each other by at least 20 degrees, such as at least 30 degrees, such as at least 45 degrees. In other examples, the first and second directions can have a different directional characteristic from each other. For instance, one directional characteristic can be a substantially straight and linear direction. Another directional characteristic can be a substantially curved direction, such as a helical or circular orientation. Other directional characteristics can define any suitable geometry as desired so as to provide a directional reinforcement along the direction of the directional characteristic.

While each reinforced region 100 can include a plurality of reinforcement strands 104 that are oriented in the respective directions, it is appreciated that each reinforced region 100 can alternatively include a single reinforcement strand 104. The single reinforcement strand 104 can extend along a single path substantially in the respective direction. Alternatively, the single reinforcement strand 104 can be folded about itself as many times as desired so as to extend back and forth along the several paths in respective direction as desired. Thus, each reinforced region 100 can be said to include at least one reinforcement strand 104. The at least one reinforcement strand 104 is present in sufficient quantity to provide for the increased strength characteristics as described herein. In one example, the at least one reinforcement strand 104 can be present in each reinforced region 100 in a range from approximately 10% to approximately 80% by volume, such as from approximately 25% to approximately 70% by volume, including from approximately 35% to approximately 65% by volume.

It is recognized that while the reinforced regions 100 can include the reinforcement strands 104 that are oriented substantially in the direction of the load during use, the reinforced regions 100 can include additional auxiliary strands in addition to the reinforcement strands as desired. Alternatively, the reinforcement strands 104 can be the only strands at the reinforced region 100, such that the reinforced region is devoid of auxiliary strands. In examples whereby the reinforced region 100 includes auxiliary strands, the auxiliary strands can also be embedded in the solidified polymer 106, and can be oriented in any direction as desired. The auxiliary strands can be made of the same material or different material than the reinforcement strands 104. Further, the auxiliary strands can have any suitable shape as desired. It may be desired to include auxiliary strands, for instance, if it is anticipated that multiple forces in different directions can be applied to the reinforced region 100. A strand can be considered to be an auxiliary strand if it is angularly offset at least 20 degrees with respect to the reinforcement strands. In one example, less than half of all strands in each of the reinforced regions 100 are oriented along a direction that is at least 20 degrees angularly offset with respect to the respective direction of the reinforcement strands 104. For instance, in some examples, less than 25% of all strands in each of the reinforced regions 100 are oriented along a direction that is at least 20 degrees angularly offset with respect to the respective direction of the reinforcement strands 104. It should be appreciated that the implant body 260, and thus the anatomical implant 220, can be constructed having any suitable alternative size, shape, and configuration as desired, and all such alternatives are contemplated herein.

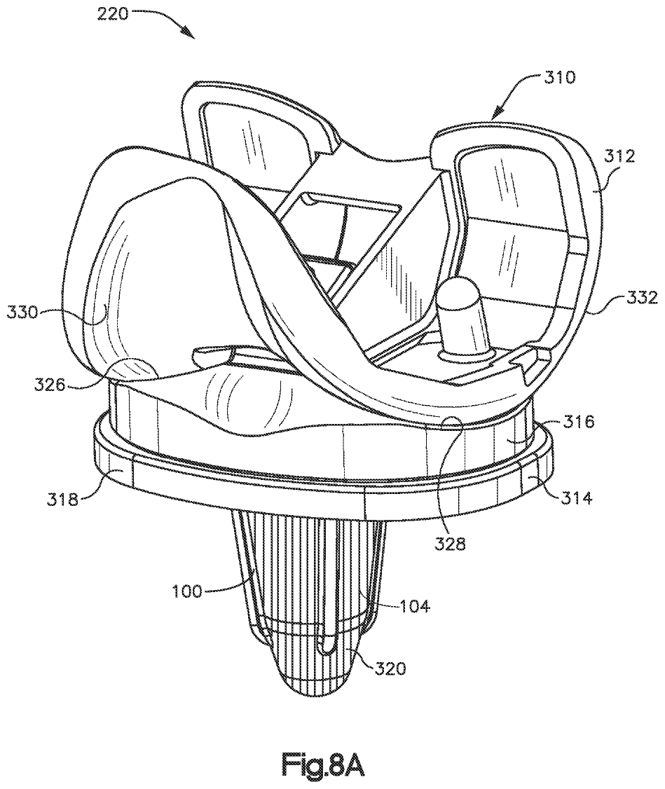

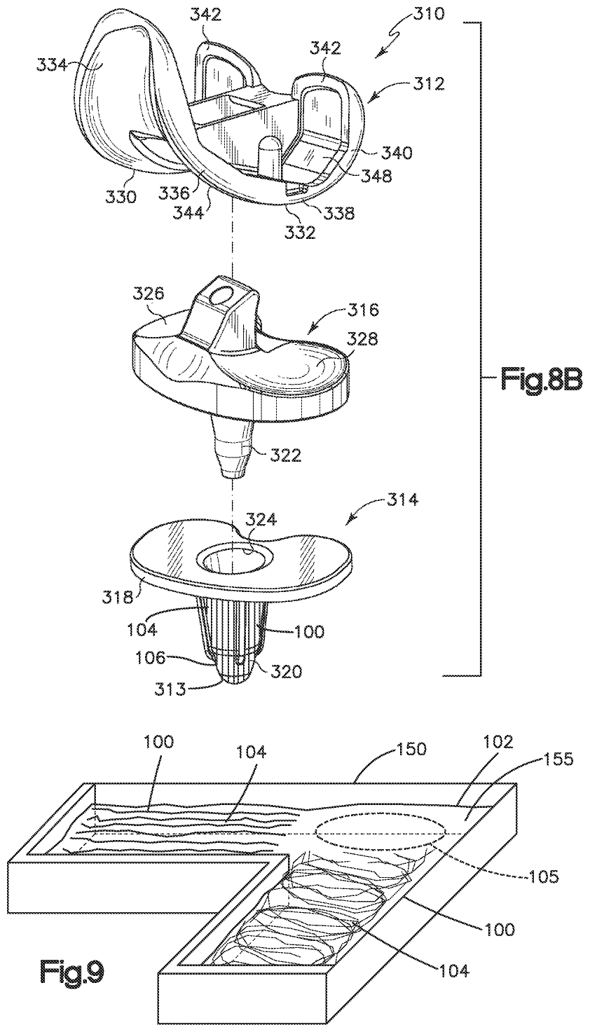

Referring now to FIGS. 8A-8B, the anisotropic anatomical implant 220 can be configured as an articulating implant, such as a knee prosthesis 310. The knee prosthesis 310 includes a femoral component 312, a tibial tray 314, and a bearing 316 configured to be coupled to the tibial tray 314. The implant body 260 having at least one reinforced region 100 can be defined by any one or more up to all of the femoral component 312, the tibial tray 314, and the bearing 316. The femoral component 312 is configured to be secured to a surgically-prepared end of a patient's distal femur (not shown), whereas the tibial tray 314 is configured to be secured to a surgically-prepared end of a patient's proximal tibia (not shown).

The tibial tray 314 includes a platform 318 having a fixation member, such as an elongated stem 320, extending away from its lower surface. The bearing 316 includes a stem 322 (see FIG. 2) that is positionable within a complementary bore 324 (see FIG. 2) in the tibial tray 314. In this manner, the bearing 316 rotatable relative to the tibial tray 314. In other embodiments, the bearing 316 may be snap-fit or otherwise secured to the tibial tray 314. Thus, the bearing 316 can fixed relative to the tibial tray 314, such that the bearing 316 is not rotatable or otherwise moveable in the anterior/posterior or medial/lateral directions with respect to the tibial tray 314. It should be appreciated that in such embodiments, other fixation members 313, such as one or more short pegs or posts, may be used in lieu of the elongated stem 320. In other examples, the tibial tray 314 can define the bearing 316 which can be monolithic with the tibial tray 314. In some examples, the platform 318 can define the bearing 316.

The bearing 316 includes at least one articular surface such as a lateral articular surface 326 and a medial articular surface 328. The articular surfaces 326 and 328 are configured to articulate with a lateral condyle surface 330 and a medial condyle surface 332, respectively, of the femoral component 312. Specifically, the femoral component 312 can be configured to emulate the configuration of the patient's natural femoral condyles, and, as such, the lateral condyle surface 330 and the medial condyle surface 332 are configured (e.g., curved) in a manner which mimics the condyles of the natural femur. The lateral condyle surface 330 and the medial condyle surface 332 are spaced apart from one another thereby defining an intercondylar notch therebetween. The bearing 316 may be constructed with a material that allows for smooth articulation between the bearing 316 and the femoral component 312, such as a polymeric material. One such polymeric material is polyethylene such as ultrahigh molecular weight polyethylene (UHMWPE), although other biocompatible polymers may be used.

The components of the knee prosthesis 310 that engage the natural bone, such as the femoral component 312 and the tibial tray 314, may be constructed with a biocompatible metal such as a cobalt chrome alloy, or a polymer 306 of the type described above. Thus, the implant body 260 defined by the articulating implant can include a reinforced region 100 that can include a resin 102 (see FIG. 9) that has been solidified so as to define a polymer 106, and at least one reinforcement strand 104 such as a plurality of reinforcement strands 104 that are disposed in the polymer 106.

Although the femoral component 312 is herein illustratively described as a monolithic component, it is characterized by a number of “regions” or “structures”. For example, the anterior structure of the femoral component 312 is referred to as an anterior flange 334. The anterior flange 334 transitions to an anterior chamfer region 336, which, in turn, transitions to a distal condylar region 338. The distal condylar region 338 transitions to a posterior chamfer region 340. A pair of posterior femoral condyles 342 form the posterior structure of the femoral component 312.

As shown, both the lateral condyle surface 330 and the medial condyle surface 332 are formed in the articular side 344 of the femoral component 312. A fixation side 348 is opposite the articular side 344, and is the side of the femoral component 312 that contacts the surgically-prepared distal femur of the patient. The fixation side 348 includes multiple surfaces that mate with planar surfaces surgically cut into the patient's distal femur.

During a surgical procedure to implant the femoral component 312 to the surgically-prepared distal end of the patient's femur. The femoral component 12 is then positioned on the patient's surgically-prepared distal femur, which has also been coated in bone cement. The femoral component 312 can be bonded to the distal femur with any suitable bone cement.

In one example, the fixation member 313 of the tibial tray 314 can define the anisotropic implant body 260. Thus, the fixation member 313 can be made from a polymer 106 having the reinforcement strands 104 so as to define a reinforced region 100. A portion up to an entirety of the fixation member 313 can define the reinforced region 100. The reinforcement strands 104 can be oriented substantially along a direction that is included in a plane along which the tibial tray 314 articulates with respect to the femoral component 312. In one example, the direction can be parallel to the direction of elongation of the fixation member 313. In other examples, the direction can be angularly offset with respect to the direction of elongation of the fixation member 313. The implant 220 is further described in U.S. Pat. No. 8,287,601, the disclosure of which is hereby incorporated by reference as if set forth in its entirety herein.

While the implant 220 has been illustrated as a knee prosthesis, it should be appreciated that the implant 220 can alternatively be configured as any other articulating implant, such as a hip prosthesis, a shoulder prosthesis, an elbow prosthesis, or the like. Thus, the implant 220 can define a ball and socket joint, and the anisotropic implant body 260 can be defined by any first member of the implant 220 that is configured to be secured to bone, and that has an articular surface that articulates with respect to a second member of the implant. The first member can define a ball or the socket of the joint, and the second member can define the other of the ball or the socket of the joint. The first member can define a connector that is configured to be secured to bone, and the connector can define the anisotropic implant body 260 in the manner described above. The ball and socket joint can be a spherical joint, or can be a round non-spherical joint. The orientation of the reinforcement strands 104 can lie on the plane of articulation of the first member of the implant. Accordingly, the plane of articulation of the first member of the implant can be at least partially defined by the orientation of the reinforcement strands 104.

Referring now to FIG. 9, one method for fabricating an anisotropic surgical implement body 155 will be described, where the implement body 155 can be configured as the instrument body 48 of the anisotropic surgical instrument 20 in accordance with any example described herein and/or the implant body 260 of the anisotropic anatomical implants 200 in accordance with any example described herein. The method for fabricating the implement body 155 can include the step of positioning the reinforcement strands 104 in a mold cavity 150, and introducing a viscous resin 102 into the mold cavity 150. The one or more auxiliary strands can also be placed into the mold cavity 150 as desired, and positioned as described herein. The reinforcement strands 104 are positioned to define one or more of the reinforced regions 100 and one or more of the weakened regions 105 as described above. Thus, the reinforcement strands 104 can be oriented along the substantially linear and straight direction at one region of the implement body 155, and the reinforcement strands 104 can be oriented along the curved path at another region of the implement body 155 that is separated by a weakened region as shown. However, it should be appreciated that the reinforcement strands 104 can be alternatively oriented as desired, and positioned to define any number of reinforced regions 100 and weakened regions 105 as desired. The reinforcement strands 104 can be positioned in the mold cavity 150 prior to introducing the viscous resin 102 into the mold cavity 150. Alternatively, the reinforcement strands 104 can be positioned in the mold cavity 150 after introducing the viscous resin 102 into the mold cavity 150. The viscous resin 102 can then solidify so as to define the polymer 106 described above. The resin 102 and resulting solidified polymer 106 can be an ultra high molecular weight (UHMW) polyethylene, poly-ether-ether-ketone (PEEK), or any suitable alternative polymer as desired.

It is recognized that alternative methods are available for fabricating the implement body 155, and the present disclosure is intended to encompass all such methods. By way of example, and not limitation, one such alternative method is to 3D print the implement body 155. For instance, the at least one reinforcement strand 104 can be fabricated using fused filament fabrication (FFF) whereby filaments of the at least one reinforcement strand 104 are fused together to fabricate the at least one reinforcement strand 104. The at least one reinforcement strand can be encapsulated in the polymer as desired. FFF can similarly be used to fabricate the one or more auxiliary strands as desired. In another option, at least one reinforcement strand 104 and one or more auxiliary strands can be included in the implement body 155 using methods described in U.S. Pat. No. 10,730,236. The disclosure of U.S. Pat. No. 10,730,236 is hereby incorporated by reference as if set forth in its entirety herein.

It should be appreciated that the illustrations and discussions of the embodiments shown in the figures are for exemplary purposes only, and should not be construed limiting the disclosure. One skilled in the art will appreciate that the present disclosure contemplates various embodiments and examples. Additionally, it should be understood that the concepts described above with the above-described embodiments and examples may be employed alone or in combination with any of the other embodiments and examples described above. It should be further appreciated that the various alternative embodiments and examples described above with respect to one embodiment can apply to all embodiments and examples as described herein, unless otherwise indicated.

What is claimed:

1. An anisotropic surgical instrument comprising:
a first reinforced region including a first at least one reinforcement strand that is embedded in a solidified polymer,
a weakened region that includes the solidified polymer and one of:
i) none of the first plurality of strands embedded in the solidified polymer; and
ii) a density of the first plurality of strands less than that of the first reinforced region.

2. The anisotropic surgical instrument of claim 1, wherein the first reinforced region is a first directionally reinforced region whereby the first at least one reinforcement strand is oriented substantially along a first direction that is oriented substantially along a direction of a force applied to the first reinforced region during use.

3. The anisotropic surgical instrument of claim 2, wherein the first at least one reinforcement strand is oriented substantially along a first direction that is oriented substantially along a direction of a primary force applied to the first reinforced region during use.

4. The anisotropic surgical instrument of claim 2, wherein the weakened region includes at least one strand oriented in a direction other than the first direction.

5. The anisotropic surgical instrument of claim 4, wherein the weakened region is devoid of any reinforcement strands embedded in the solidified polymer.

6. The anisotropic surgical instrument of claim 3, wherein the solidified polymer of the weakened region is monolithic and unitary with the solidified polymer of the first directionally reinforced region.

7. The anisotropic surgical instrument of claim 3, wherein the first at least one reinforcement strand comprises a first plurality of reinforcement strands.

8. The anisotropic surgical instrument of claim 2, wherein the first direction is substantially straight and linear.

9. The anisotropic surgical instrument of claim 2, comprising a handle portion and a torsional driving portion that is defined by the first directionally reinforced region, wherein the torsional driving portion is configured to deliver a torsional force along a central axis, and the first direction defines a substantially curved path about the central axis.

10. The anisotropic surgical instrument of claim 9, wherein the curved path substantially defines a helix.

11. The anisotropic surgical instrument of claim 9, wherein the torsional driving portion comprises a reamer that is rotatable with respect to the handle.

12. The anisotropic surgical instrument of claim 9, wherein the torsional driving portion comprises a screw driving head.

13. The anisotropic surgical instrument of claim 1, wherein at least some of the first at least one reinforcement strand comprises carbon.

14. The anisotropic surgical instrument of claim 1, wherein at least some of the first at least one reinforcement strand comprises glass.

15. The anisotropic surgical instrument of claim 1, wherein at least some of the first at least one the reinforcement strand is fibrous.

16. An anisotropic surgical instrument comprising:
a shaft that extends along a central axis, from a proximal end to a distal end, and a head that extends from the distal end,
wherein at least a portion of the shaft includes a solidified polymer and at least one reinforcement strands that is embedded in the solidified polymer and oriented substantially helically about the central axis, and
wherein the shaft is configured to rotate such that the head transmits a torsional force.

17. The anisotropic surgical instrument of claim 16, wherein the head defines a reamer head.

18. The anisotropic surgical instrument of claim 16, wherein the head defines a screw driving head.

19. The anisotropic surgical instrument of claim 18, wherein the screw driving head defines a cruciform.

20. The anisotropic surgical instrument of claim 19, wherein the screw driving head defines a socket.
